(19) United States
(12) Patent Application Publication
(10) Pub. No.: US 2006/0276861 A1
(43) Pub. Date: Dec. 7, 2006

(54) Non-invasive method and system for the treatment of snoring and nasal obstruction

(76) Inventor: J. T. Lin, Oviedo, FL (US)

Correspondence Address: J. T. Lin, 4532 Old Carriage Trail, Oviedo, FL, 32765, US

(21) Appl. No.: 11/140,904

(22) Filed: Jun. 1, 2005

(51) Int. Cl. A61N 5/06; A61B 18/18

(52) U.S. Cl. 607/89; 606/2

(57) ABSTRACT

Laser for thermal shrinkage of soft tissue of uvula, soft palate, nasal turbinate or tongue base for the treatment of snoring, nasal obstruction or sleep apnea are disclosed. The preferred laser includes infrared laser about 0.7 to 1.85 micron, pulse duration about 100 microsecond to 5 seconds, spot size of about 2 to 5 mm and power of about 2 to 20 W at the treated area. The laser energy is delivered to the treated area by an optical fiber and a hand piece to cause a localized temperature about 65 to 85 degree Celsius for sufficient shrinkage of the treated soft tissues. Optical fiber bundles to produce high-power diode laser output or multi-wavelength are also disclosed.

### BACKGROUND OF THE INVENTION

1. Field of the Invention

This invention relates to method and system for the treatment of snoring, sleep apnea and nasal obstruction, particularly for shrinkage of soft tissue by a laser.

2. Prior Art

Snoring is caused by irregular air flow of the nose which results in non-controllable vibration of the uvula or soft palate. Various methods have been used to treat snoring. These prior arts include the use of anti-snoring solution, such as U.S. Pat. No. 6,790,465, or anti-snoring device such as U.S. Pat. No. 6,748,951. Bipolar cautery and radio frequency (RF) somnoplasty have been used commercially, which requires the use of needle electrode and delivery of 200 to 500 J in each treated spot. Surgical method using a carbon dioxide laser has been used to remove (ablate) a portion of the uvula or palate soft tissue. This prior art (ablative surgery) is invasive, painful having delayed bleeding or synechia formation, and requires a long healing time and it is not recommended for sleep apnea.

Thermal lasers have been used for the treatment of hyperopia, such as U.S. Pat. No. 5,484,432, using wavelength of 1.8 to 2.2 micron and a shallow absorption depth of the corneal tissue about 0.45 mm. There is no commercially available thermal laser for snoring treatment which requires a much deeper depth about 2 to 5 mm.

One objective of this invention is to provide a non-invasive laser method and system to obviate drawbacks of prior arts and improve the treatment efficacy.

It is yet another objective of this invention is to define the optimal laser parameters and the area and depth for various soft tissues to be treated for the treatment of snoring, sleep apnea and nasal obstruction.

It is yet another objective of this invention is to include the disclosure of integrated system design including optical fiber delivery, focusing optics and multi-wavelength mixture.

It is yet another objective of this invention is to include the disclosure of the laser tissue interaction mechanism behind the treatment, for the criteria of wavelength selection.

In comparing to a RF device, the laser method of this invention offers the following advantages: less invasive, much smaller energy (about 5 to 50 J) is needed in each treated spot; faster procedure using adjustable laser spot size of 1 to 5 mm (versus a penetrating needle about 0.5 mm in RF); both contact and non-contact mode treatment (versus a penetrating needle in RF device); penetration depth controllable by laser wavelength selected; and multi-wavelength laser output for optimal outcome (not available in RF device).

### SUMMARY OF THE INVENTION

The preferred embodiment of this invention includes the laser shrinkage of uvula or soft palate (for snoring), tongue base (for sleep apnea) or nasal turbinate (for nasal obstruction).

It is yet another preferred embodiment is that the treated area is locally heated without damaging the surrounding tissue, where the localized temperature is raised to about 65 to 85 degree Celsius (C.), most preferable about 75 to 80 degree C., to cause efficient thermal shrinkage of the treated area

It is yet another preferred embodiment includes a heating penetration depth on the treated area about 2 to 5 mm governed by the power and wavelength of the laser and treated tissues.

It is yet another preferred embodiment includes a fiber-delivered laser beam applied to the treated area in either contact or non-contact mode.

It is yet another preferred embodiment includes a laser having a wavelength in the infrared of 0.7 to 1.85 microns, such as semiconductor laser (0.7 to 1.85 microns), Nd:glass (at 1.54 micron), Nd:YAG or Nd:YLF (at 1.3 or 1.4 micron); pulse width of 100 microsecond to about 5 seconds, or operated at free-running normal mode, or a continuous wave (CW).

It is yet another preferred embodiment includes a fiber bundle having the same wavelength diode or 2 to 3 different wavelengths selected from a group consisting of about 0.8, 0.9, 0.94, 0.98, 1.3, 1.45, 1.54 and 1.85 microns.

Further preferred embodiments of the present invention will become apparent from the description of the invention that follows.

### BRIEF DESCRIPTION OF THE DRAWINGS

FIG. 1 Schematics of treated area.

FIG. 2 Schematics of laser system and delivery unit.

FIG. 3 Schematics of fiber-coupled unit.

FIG. 4 Schematics of non-contact and contact laser energy beam.

FIG. 5 Schematics of focused beam from a fiber tip.

### DETAILED DESCRIPTION OF THE INVENTION AND THE PREFERRED EMBODIMENTS

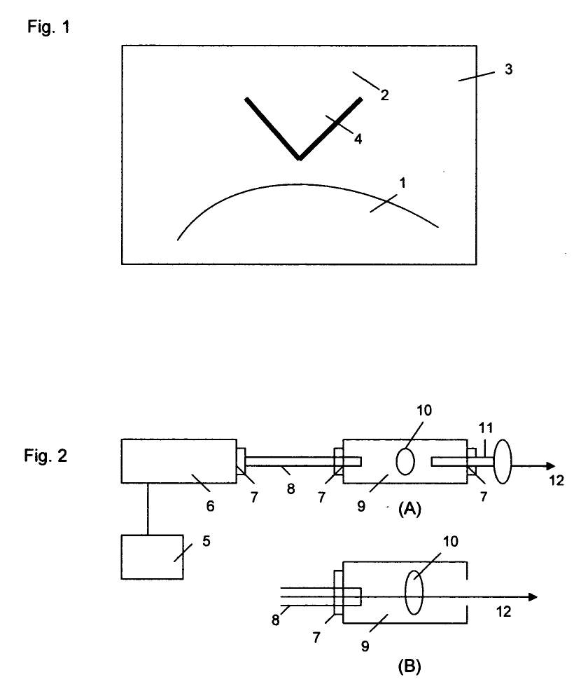

As shown in FIG. 1, when one falls into a deep sleep, the muscles in the tongue 1, throat and root of the mouth, the soft palate 2, relax. This muscle relaxation causes the throat tissues to sag and narrow the airway of the mouth 3, creating the sound of snoring. Snoring may be also caused a longer-than-normal uvula 4, thick soft palate or enlarge of tonsils or adenoids tissue between the back of the nose and throat. When one sleeps on back, the tongue falls backwards into the throat, this may also narrow the airway and partly block airway. Sleep apnea is the period when one stops breathing while one is sleeping, which may last for 10 seconds or longer. Both snoring and sleep apnea are caused by the throat tissues relaxation. Overweight or older people with weaker (or less firm) throat muscles is other factor causing snoring or sleep apnea. Nasal obstruction is due to chronic turbinate enlargement (or hypertrophy). Therefore, the preferred embodiment of this invention is to use laser energy to cause the shrinkage of the uvula, soft palate, tongue base or nasal turbinate and widen the airway to reduce or stop snoring or sleep apnea.

When a laser is used, we also require efficient localized tissue heating with minimal thermal damage to the non-treated tissue. Therefore, the preferred laser spectrum of this invention is the region where the treated tissues including soft palate, uvula, nasal turbinate or tongue base (containing blood, melanin or water) have certain absorption, but not too strong, in order to penetrate deep into the selected area for maximal shrinkage. Based on these criteria, the preferred laser spectrum includes infrared (IR) laser at about 0.7 to 1.85 microns. Other ranges of spectrum with very strong tissue absorption such as carbon dioxide laser (at 10.6 microns) or other IR laser about 1.9 to 2.2, or about 2.8 to 3.2 microns, visible laser of 0.4 to 0.69 microns or UV laser of 193 to 300 nm should be excluded. These ‘ablation-type” lasers, excluded in the present invention, are required in the prior arts which use laser to remove (ablate) tissues, rather than thermal shrinking. For lasers in the above selected IR range, the preferred pulsed duration is longer than 100 microseconds, or a continuous wave (CW) mode at low peak power (less than 500 W), comparing to the prior arts of ablation procedure which requires very high peak power (over 100 KW).

The preferred lasers of this invention include solid-state or diode lasers at about 0.7 to 1.85 microns. The most preferable laser spectra are at about 0.75, 0.8, 0.94, 0.98, 1.3, 1.45, 1.54 and 1.85 microns from semiconductor diode lasers, or Nd:glass (at 1.54 micron), or Nd;YAG or Nd:YLF (at 1.3 or 1.4 micron). The preferred laser pulse width is about 100 microseconds to 5 second, operated at CW or quasi-CW mode.

The preferred embodiment of this invention further includes the use of multiple spots on each of the treated area, where each spot also includes multiple pulses of about 1 to 10. It also includes a multiple treatments of about 1 to 5, depending on area treated and applications, over a period of about 2 to 10 weeks. In comparison, when a RF device is used, a typical energy delivery time for each spot is about 80 to 200 seconds which is much longer than that of a laser is used in this invention (about 5 to 30 seconds). In addition, only about 5 to 50 J laser energy is needed in each treated spot for each treatment, which is much smaller than 200 to 500 J of RF energy when a RF device is used.

It was previously known (for example: Bargeon et al. “Calculated and measured endothelial temperature histories of excised rabbit cornea explored to IR radiation”, Exp. Eye Research, vol. 32, 241-250, 1981; Stringer et al. “Shrinkage temperature of eye collagen”, Nature, vol. 204, 1307, 1964) that collagen fiber may contract to about one-third of their linear dimension when it is heated to about 60 to 70 degree Celsius. This thermal shrinkage in corneal tissue shall also occur similarly to the treated soft tissues proposed in this invention. In Lin's proposed “laser induced” thermal shrinkage (LTS), there is a minimal amount of thermal energy needed in order to cause sufficient LTS. LTS is further governed by the localized temperature (T) of the treated tissue. Depending on the types of soft tissue (uvula, nasal turbinate, palate or tongue), the preferred T=(65 to 85) degree Celsius, most preferable of 75 to 80 degree Celsius, and shall not be too high to cause permanent tissue damage or evaporation. Given a laser energy (E), T is proportional to W=Et, where t is the laser treating time and W is the average power (in Watt) applied to the tissue. To cause effective LTS of the tissue, only those lasers with appropriate spectra can be used, such that the laser energy can be localized absorbed by the treated tissue via the melanin, blood or water content of the tissue.

We note that without the above theoretical analysis, it would be very difficult to predict the clinical outcome. Our method in this invention and the parameters for the proposed device and clinical techniques are based upon the above analysis relating to the absorption depth, and temperature required for efficient shrinkage.

As shown in FIG. 2(A), a microprocessor (or computer) 5 is used to control the parameters of the laser unit 6, coupled by a standard connector 7 to an optical fiber 8, further connected to an fiber end piece 11 to deliver the laser output beam 12 to the treated area. The hand piece 9, shown in FIG. 2 (B), may consist of a collimating lens 10 to convert the divergent beam from the fiber 8 into a significantly collimated beam 12. The spot size and shape of the output beam 12 may be altered by its location and by using different focal length (f) of the lens 10 or more than one optics. Therefore, lens 10, in general, is a means of beam spot and shape control consisting of one or more optics inside the hand piece. The preferred focal length includes about 2 to 20 mm and hand piece length is about 10 to 20 cm having a diameter about 5 to 10 mm. The preferred optical fiber includes material highly transparent to the selected IR laser beam and having a length about 1.0 to 1.5 meter and flexible. This fiber is commercially available.

The preferred lasers of this invention include solid-state or diode lasers at about 0.7 to 1.85 microns. The most preferable laser spectra are at about 0.8, 0.9, 0.94, 0.98, 1.3, 1.45, 1.54 and 1.85 microns from semiconductor diode lasers, or Nd:glass (at 1.54 micron), or Nd;YAG or Nd:YLF (at about 1.3 or 1.4 micron). The preferred laser pulse width is about 100 microseconds to 5 second, operated at CW or quasi-CW mode. The preferred energy beam spot size (in non-contact mode) or the size of the fiber tip (for contact mode) is about 2 to 5 mm on the treated surface. The preferred average power at each of the treated spot is about 2 to 20 W, depending on spot size, spectra and power of the laser beam and the types of tissues treated. For example, a power of 20 W needed for a spot size of 5 mm will be reduced to about 3 W when a small spot of 2 mm is used. This is based on our theory that the laser-induced temperature increase of the soft tissue is proportional to the fluency (F) times the treated period of each spot, where F is the energy per unit area and area is proportional to square of the spot diameter. Greater details will be disclosed later.

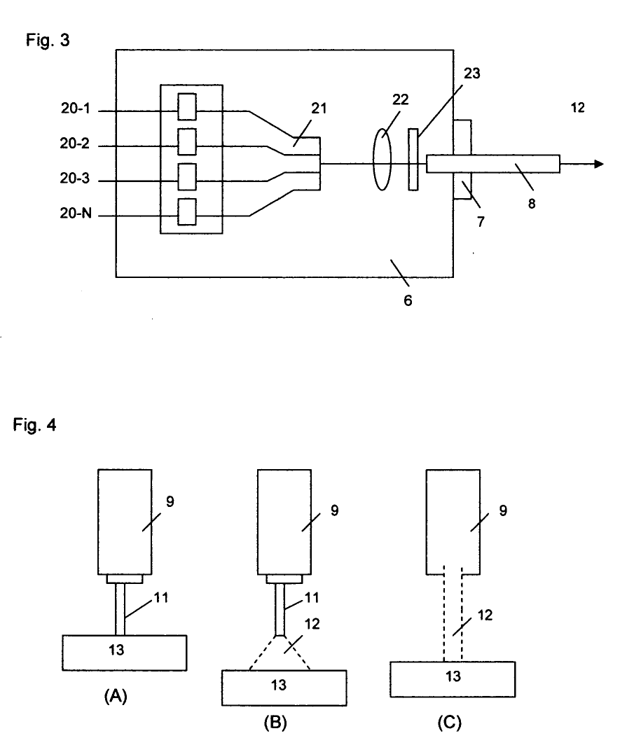

FIG. 3 shows a schematics of a diode laser unit 6, consists of a series of diode single chips or arrays 20-1 to 20-N which are combined in a fiber bundle 21 and re-coupled to a single fiber 8 by a focusing lens 22 and an alignment optics 23 ( a pair of 45 degree angle high-reflecting optics) to produce an output beam 12 having a power of about NPi, Pi being the power of the single chip (or array). This preferred embodiment allows us to produce a higher power diode laser by combining a set of small power single source. Furthermore, a visible aiming beam (e. g., a red diode at about 630 nm) may also be integrated into the fiber bundle 21. For the case of single array, the preferred focusing lens 22 also includes a spherical or cylinder lens or their combination such that the linear beam from the array may be reshaped into a circular spot at the entrance of the fiber 8.

The preferred diode laser chips or arrays shown in FIG. 3 includes that all of them having the same wavelength, or a combination of 2 to 3 wavelengths selected from the IR spectrum of 0.7 to 1.85 micron, or the most preferable spectrum of about 0.8, 0.9, 0.94, 0.98, 1.3, 1.45, 1.54, 1.7 and 1.85 microns. This multi-wavelength mixture can be easily achieved by the above described fiber bundle, but would be difficult to do, otherwise.

FIG. 4 (A) to 4 (C) show the preferred embodiments of laser interaction with the treated tissue 13, in a contact mode (A) fiber tip 11 contact with the treated surface, whereas (B) and (C) are non-contact modes with a divergent beam (B) and a collimated beam (C).

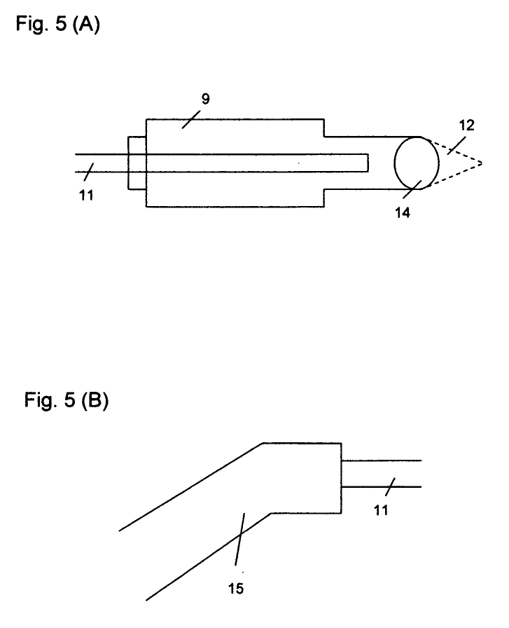

Another preferred embodiment of this invention includes that, as shown by FIG. 5(A) for a preferred contact mode, the end of the fiber tip 11 is further coupled to a focusing lens 13 such that the laser beam energy can penetrate deep into the treated area for maximal tissue shrinkage. Alternatively, the focusing beam may be produced by a fiber tip having a round shape as shown in 5 (B) which also shows one preferred shape of the curved end piece 15 that allows the surgeon to observe and locate the treated area. The preferred curved angle is about 30 to 70 degree.

Another preferred embodiment of this invention includes that the output beam 12 having a spot of about 2 to 5 mm is used to treat (using multiple spots) an area of about 5 to 15 mm of the soft palate, uvula or tonsils.

The preferred embodiment of this invention includes that the diode array having the same wavelength or a combination of more than one wavelength selected from a group of wavelength in the IR about 0.7 to 1.85 microns depending on the treated areas. For deeper laser penetration, wavelength shorter than about 1.3 micron is preferred, and for shallow laser penetration, stronger absorption spectra of about 1.4 to 1.85 microns are preferred. The preferred laser penetration depth includes about 0.5 to 8 mm (most preferable of about 1 to 2 mm) for thermal shrinkage of soft palate, 2 to 3 mm for uvula, and 2 to 4 mm for tongue base or nasal turbinate. We note that the degree of shrinkage (or volume reduction of the treated area) is proportional to the volume (area x depth) of laser penetration, or the volume (area) where the temperature profile above the shrinkage threshold temperature, about 55 to 60 degree Celsius. The preferred laser penetration depths (PD) for various treated tissues disclosed in this invention (governed by the laser wavelength) are based on clinically preferred condition of maximal depth with a minimal pain. For example, soft palate is thinner than other tissues, but needs a larger treated area, therefore, shallower PD is preferred. In comparison, uvula has a smaller, but thicker area, which needs a deeper PD to achieve effective shrinkage.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus, the ophthalmic applications herein disclosed are to be considered merely as illustrative and the invention is to be limited only as set forth in the claims.

I claim:

1. A method of thermal shrinkage of soft tissue, comprising the steps of:
(a) selecting a laser beam having a predetermined power, spot size and wavelength; and
(b) delivering said laser beam to said soft tissue of a predetermined treated area, whereby patient's snoring, sleep apnea or nasal obstruction is treated.

2. A method of claim 1, wherein said treated area includes the uvula, nasal turbinate, soft palate or tongue base.

3. A method of claim 1, wherein said laser beam includes a laser having a wavelength of about 0.7 to 1.85 micron, a pulse duration about 100 microsecond to 5 seconds, a spot size of about 2 to 5 mm, and a power of about 2 to 20 W at each spot of said treated area in each treatment.

4. A method of claim 1, wherein said laser beam includes Nd:YAG , or Nd:YLF laser at about 1.3 or 1.4 micron, or Nd:glass at about 1.54 micron, or semiconductor laser at about 0.7 to 1.85 micron.

5. A method of claim 1, wherein said laser beam energy is delivered to said treated area by an optical fiber and a hand piece.

6. A method of claim 5, wherein said hand piece consists of a means of beam spot size and shape control including one or more than one focusing lens having a focal length of about 2 to 20 mm.

7. A method of claim 1, wherein said laser beam energy is delivered to said treated area to cause a localized temperature of about 65 to 85 degree Celsius, most preferable about 70 to 80 degree Celsius, and a thermally treated depth of about 0.5 to 5 mm.

8. A method of claim 1, wherein said laser beam interacts with said treated area in a non-contact mode, or a contact mode using a focused or collimated beam.

9. A system for the treatment of snoring, nasal obstruction, or sleep apnea consisting of:
(a) a laser beam having a predetermined power, spot size and wavelength; and
(b) a delivering means to deliver said laser beam to soft tissue of a predetermined area, whereby said soft tissue is thermally shrunk by said laser beam energy for the treatment of snoring, sleep apnea or nasal obstruction.

10. A system of claim 9, wherein said predetermined area includes the uvula, nasal turbinate, soft palate or tongue base.

11. A system of claim 9, wherein said laser beam includes a laser having a wavelength of about 0.7 to 1.85 micron, pulse duration about 100 microsecond to 5 seconds, a spot size of about 2 to 5 mm, and a power of about 2 to 20 W at said predetermined area for each treatment.

12. A system of claim 9, wherein said laser beam includes Nd:YAG , or Nd:YLF laser at about 1.3 or 1.4 micron, or Nd:glass at about 1.54 micron, or semiconductor laser at about 0.7 to 1.85 micron.

13. A system of claim 9, wherein said laser beam energy is delivered to said treated area by an optical fiber and a hand piece.

14. A system of claim 13, wherein said hand piece consists of one or more than one focusing or collimating lens having a focal length of about 2 to 20 mm.

15. A system of claim 13, wherein said optical fiber is made of a material highly transparent to said laser beam wavelength 0.7 to 1.9 microns, flexible and having a length about 1.0 to 1.5 meters.

16. A system of claim 9, wherein said laser beam includes laser diode arrays combined into a fiber bundle and re-coupled to a single fiber having an output power equals the summation of the power of single arrays.
